(19) United States
(12) Patent Application Publication
(10) Pub. No.: US 2025/0111946 A1
(43) Pub. Date: Apr. 3, 2025

(54) INDEPENDENCE SUPPORT METHOD, INFORMATION PROCESSING APPARATUS, SYSTEM, AND PROGRAM

(71) Applicant: Sompo Care Inc., Shinagawa-ku, Tokyo (JP)

(72) Inventors: Naoki HIRANUMA, Shinagawa-ku, Tokyo (JP); Shigeki KAWABATA, Shinagawa-ku, Tokyo (JP); Midori MASUYAMA, Shinagawa-ku, Tokyo (JP); Yukiko OE, Shinagawa-ku, Tokyo (JP)

(73) Assignee: Sompo Care Inc., Shinagawa-ku, Tokyo (JP)

(21) Appl. No.: 18/838,338

(22) PCT Filed: Feb. 22, 2023
(86) PCT No.: PCT/JP2023/006597

§ 371 Date: Aug. 14, 2024

(30) Foreign Application Priority Data
Mar. 3, 2022 (JP) 2022-032939

(51) Int. Cl. G16H 50/30; G16H 10/60; G16H 15/00

(52) U.S. Cl. CPC G16H 50/30; G16H 10/60; G16H 15/00

(57) ABSTRACT

Technology related to independence support is improved. An independence support method to be executed by an information processing apparatus includes determining a health care score of a subject, predicting a health status of the subject after a first predetermined period based on the health care score, predicting a health status of the subject after a second predetermined period based on a transition performance of a health status of a group to which the subject belongs, the group being determined by the health care score, and presenting predictive information based on the health status after the first predetermined period and the health status after the second predetermined period.

### TECHNICAL FIELD

The present disclosure relates to an independence support method, an information processing apparatus, a system, and a program.

### BACKGROUND

Technology related to independence support has been developed. For example, Patent Literature (PTL) 1 discloses an evaluation support apparatus that supports a subject, such as an elderly person, to evaluate in a shorter time whether he or she can live independently.

### CITATION LIST

### Patent Literature

PTL 1: JP 2021-60930 A

### SUMMARY

### Technical Problem

From the perspectives of preventing the deterioration of a subject's health status and of promoting independent living, information on the future prediction of the subject's health status is useful. The technology in PTL 1 is focused, however, on improving the efficiency of the evaluation of whether the subject can live independently and does not sufficiently consider matters such as the future prediction of the subject's health status. Technology related to independence support thus has room for improvement.

In light of these circumstances, it is an aim of the present disclosure to improve technology related to independence support.

### Solution to Problem

An independence support method according to an embodiment of the present disclosure is an independence support method to be executed by an information processing apparatus, the independence support method including:
determining a health care score of a subject;
predicting a health status of the subject after a first predetermined period based on the health care score;
predicting a health status of the subject after a second predetermined period based on a transition performance of a health status of a group to which the subject belongs, the group being determined by the health care score; and
presenting predictive information based on the health status after the first predetermined period and the health status after the second predetermined period.

An information processing apparatus according to an embodiment of the present disclosure is an information processing apparatus including a controller, wherein
the controller is configured to
   determine a health care score of a subject,
   predict a health status of the subject after a first predetermined period based on the health care score,
   predict a health status of the subject after a second predetermined period based on a transition performance of a health status of a group to which the subject belongs, the group being determined by the health care score, and
   present predictive information based on the health status after the first predetermined period and the health status after the second predetermined period.

A program according to an embodiment of the present disclosure is a program configured to cause a computer to execute operations including:
determining a health care score of a subject;
predicting a health status of the subject after a first predetermined period based on the health care score;
predicting a health status of the subject after a second predetermined period based on a transition performance of a health status of a group to which the subject belongs, the group being determined by the health care score; and
presenting predictive information based on the health status after the first predetermined period and the health status after the second predetermined period.

### Advantageous Effect

According to an embodiment of the present disclosure, technology related to independence support can be improved.

### BRIEF DESCRIPTION OF THE DRAWINGS

In the accompanying drawings:

FIG. 1 is a block diagram illustrating a schematic configuration of a system according to an embodiment of the present disclosure;

FIG. 2 is a block diagram illustrating a schematic configuration of a terminal apparatus;

FIG. 3 is a block diagram illustrating a schematic configuration of an information processing apparatus;

FIG. 4 is a diagram illustrating an example of a subject database;

FIG. 5 is a flowchart illustrating operations of the information processing apparatus according to an embodiment of the present disclosure;

FIG. 6 is a diagram illustrating an example of a user interface outputted by the terminal apparatus;

FIG. 7 is a diagram illustrating a variation of the user interface outputted by the terminal apparatus;

FIG. 8 is a diagram illustrating a variation of the user interface outputted by the terminal apparatus;

FIG. 9 is a diagram illustrating a variation of the user interface outputted by the terminal apparatus;

FIG. 10 is a diagram illustrating a variation of the user interface outputted by the terminal apparatus;

FIG. 11 is a diagram illustrating a variation of the user interface outputted by the terminal apparatus; and

FIG. 12 is a diagram illustrating a variation of the user interface outputted by the terminal apparatus.

### DETAILED DESCRIPTION

An independence support system 1 according to an embodiment of the present disclosure is described below with reference to the drawings.

Identical or equivalent portions in the drawings are labeled with the same reference signs. In the explanation of the embodiments, a description of identical or equivalent portions is omitted or simplified as appropriate.

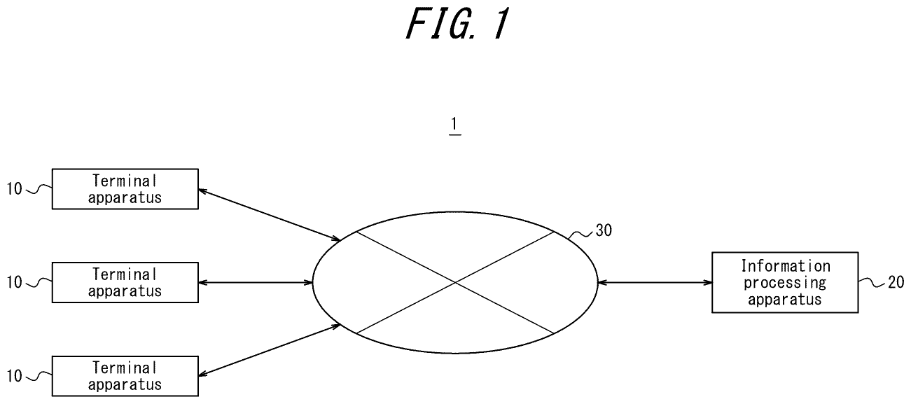

An outline and configuration of the independence support system 1 according to the present embodiment is described with reference to FIG. 1.

The independence support system 1 according to the present embodiment includes a plurality of terminal apparatus 10 and an information processing apparatus 20. The plurality of terminal apparatuses 10 and the information processing apparatus 20 are communicably connected to a network 30, such as a mobile communication network, the Internet, and the like.

The plurality of terminal apparatuses 10 is any apparatus used by each user (such as a certified care worker, caregiver, or care manager). For example, a general-purpose electronic device, such as a smartphone or tablet terminal, or a dedicated electronic device can be adopted as the terminal apparatus 10. Although FIG. 1 illustrates an example of the independence support system 1 including three terminal apparatuses 10, this example is not limiting. The independence support system 1 may include fewer than three terminal apparatuses 10 or may include four or more terminal apparatuses 10.

The information processing apparatus 20 is, for example, a server apparatus installed in a data center or the like. For example, the information processing apparatus 20 is a server belonging to a cloud computing system or other computing system. The information processing apparatus 20 can communicate with the terminal apparatus 10 via the network 30. Although FIG. 1 illustrates an example of the independence support system 1 including one information processing apparatus 20, this example is not limiting. The independence support system 1 may include two or more information processing apparatuses 20.

First, an overview of the present embodiment is described, with details to be described later. The information processing apparatus 20 determines a health care score of a subject, such as a person requiring nursing care. The health care score is determined based on the activity functions, cognitive status, and nutritional status of the subject. In the present embodiment, a person requiring nursing care is a person who needs nursing care or support in daily life. In the present embodiment, the independence support system 1 is described as being used in a residential facility where the subject resides.

The information processing apparatus 20 predicts the health status of the subject after a first predetermined period based on the health care score. Here, the health status includes the state of activity function, the state of cognitive function, and nutritional status of the subject. The first predetermined period is, for example, 3 months. The information processing apparatus 20 also predicts the health status of the subject after a second predetermined period based on the transition performance of the health status of the group to which the subject belongs, the group being determined by the health care score. The second predetermined period is, for example, 12 months. The information processing apparatus 20 presents predictive information based on the health status after the first predetermined period and the health status after the second predetermined period to a user such as a manager. The first predetermined period and the second predetermined period are not limited to 3 months and 12 months, respectively, but may be any period of time.

According to the present embodiment, the information processing apparatus 20 thus determines the health care score of the subject, predicts the health status after the first predetermined period based on the health care score, predicts the health status of the subject after the second predetermined period, and presents predictive information. In other words, according to the present embodiment, technology related to independence support is improved in that the subject's health care score can be used to present predictive information from multiple different perspectives.

Next, each of the components of the independence support system 1 will be described in detail.

### (Configuration of Terminal Apparatus)

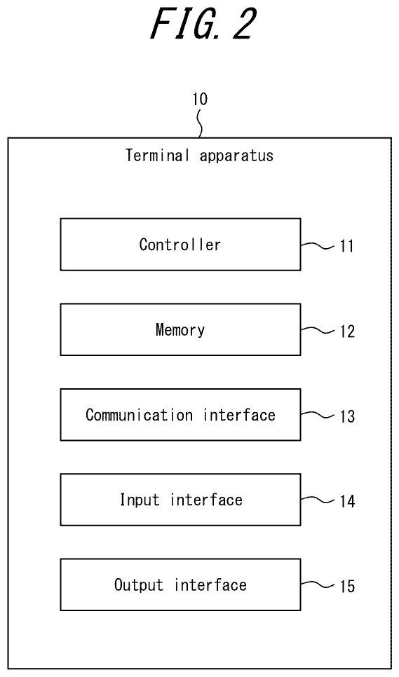

As illustrated in FIG. 2, the terminal apparatus 10 includes a controller 11, a memory 12, a communication interface 13, an input interface 14, and an output interface 15.

The controller 11 includes at least one processor, at least one dedicated circuit, or a combination thereof. The processor may, for example, be a general-purpose processor, such as a central processing unit (CPU) or graphics processing unit (GPU), or a dedicated processor specialized for particular processing. The dedicated circuit may, for example, be a field-programmable gate array (FPGA) or an application specific integrated circuit (ASIC). The controller 11 executes processing related to operations of the terminal apparatus 10 while controlling each component of the terminal apparatus 10.

The memory 12 includes at least one semiconductor memory, at least one magnetic memory, at least one optical memory, or a combination of at least two of these. The semiconductor memory is, for example, random access memory (RAM) or read only memory (ROM). The RAM is, for example, static random access memory (SRAM) or dynamic random access memory (DRAM). The ROM is, for example, electrically erasable programmable read only memory (EEPROM). The memory 12 functions as, for example, a main memory, an auxiliary memory, or a cache memory. The memory 12 stores data to be used for operation of the terminal apparatus 10 and data resulting from operation of the terminal apparatus 10.

The communication interface 13 includes at least one interface for communication with an external destination. The interface for communication may be an interface for wired communication or wireless communication. In the case of wired communication, the interface for communication may be a local area network (LAN) interface or a universal serial bus (USB), for example. In the case of wireless communication, the interface for communication may be an interface conforming to a mobile communication standard, such as Long Term Evolution (LTE), 4^{th} Generation (4G), or 5^{th} Generation (5G), or an interface conforming to short-range wireless communication such as Bluetooth® (Bluetooth is a registered trademark in Japan, other countries, or both). The communication interface 13 receives data for use in operation of the terminal apparatus 10 and transmits data resulting from operation of the terminal apparatus 10.

The input interface 14 includes at least one interface for input. The interface for input is, for example, a physical key, a capacitive key, a pointing device, or a touchscreen integrally provided with a display. The interface for input may, for example, be a microphone that receives audio input, a camera that accepts gesture input, or the like. The input interface 14 receives an operation for inputting data used in operation of the terminal apparatus 10. Instead of being provided in the terminal apparatus 10, the input interface 14 may be connected to the terminal apparatus 10 as an external input device. Any appropriate connection method can be used, such as USB, High-Definition Multimedia Interface (HDMI®) (HDMI is a registered trademark in Japan, other countries, or both), or Bluetooth®.

The output interface 15 includes at least one interface for output. The interface for output is, for example, a display that outputs information as images, a speaker that outputs information as audio, or the like. The display may, for example, be a liquid crystal display (LCD) or an organic electro luminescence (EL) display. The output interface 15 displays and outputs data resulting from operation of the terminal apparatus 10. Instead of being provided in the terminal apparatus 10, the output interface 15 may be connected to the terminal apparatus 10 as an external output device. Any appropriate connection method can be used, such as USB, HDMI®, or Bluetooth®.

The functions of the terminal apparatus 10 are implemented by a processor corresponding to the terminal apparatus 10 executing a program according to the present embodiment. In other words, the functions of the terminal apparatus 10 are implemented by software. The program causes a computer to function as the terminal apparatus 10 by causing the computer to execute the operations of the terminal apparatus 10. In other words, the computer functions as the terminal apparatus 10 by executing the operations of the terminal apparatus 10 according to the program.

The program according to the present embodiment can be recorded on a computer readable recording medium. Computer readable recording media include non-transitory computer readable recording media, examples of which are a magnetic recording apparatus, an optical disc, a magneto-optical recording medium, and a semiconductor memory. The program is, for example, distributed by the sale, transfer, or lending of a portable recording medium such as a digital versatile disk (DVD) or a compact disk read only memory (CD-ROM) on which the program is recorded. The program may also be distributed by storing the program in the storage of an external server and transmitting the program from the external server to another computer. The program may also be provided as a program product.

A portion or all of the functions of the terminal apparatus 10 may be implemented by a dedicated circuit corresponding to the controller 11. In other words, a portion or all of the functions of the terminal apparatus 10 may be implemented by hardware.

### (Configuration of Information Processing Apparatus)

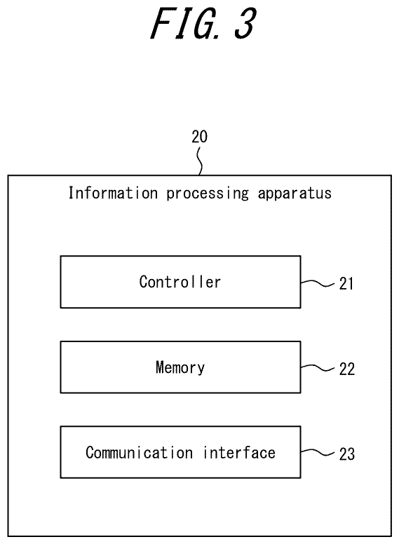

As illustrated in FIG. 3, the information processing apparatus 20 includes a controller 21, a memory 22, and a communication interface 23.

The controller 21 includes at least one processor, at least one dedicated circuit, or a combination thereof. The processor may be a general-purpose processor, such as a CPU or GPU, or a dedicated processor specialized for particular processing. The dedicated circuit is, for example, an FPGA or an ASIC. The controller 21 executes processing related to operation of the information processing apparatus 20 while controlling each component of the information processing apparatus 20.

The memory 22 includes at least one semiconductor memory, at least one magnetic memory, at least one optical memory, or a combination of at least two of these. The semiconductor memory is, for example, RAM or ROM. The RAM is, for example, SRAM or DRAM. The ROM is, for example, EEPROM. The memory 22 functions as, for example, a main memory, an auxiliary memory, or a cache memory. The memory 22 stores data to be used for operation of the information processing apparatus 20 and data resulting from operation of the information processing apparatus 20.

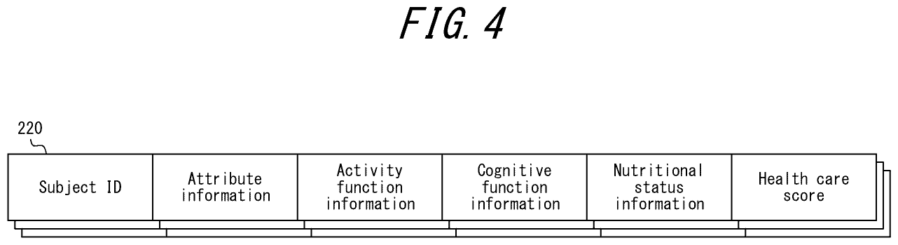

In the present embodiment, the memory 22 stores the subject database 220. FIG. 4 illustrates an example of the subject database 220. For example, the subject database 220 includes a subject ID, attribute information, activity function information, cognitive function information, nutritional status information, and a health care score.

The subject ID is information that uniquely identifies the subject. For example, the subject ID may be a number, a symbol, or a combination thereof, assigned to a resident of a nursing home, elderly care facility, or other residential facility. An example in which the subject is a resident of a residential facility is described in the present embodiment, but the subject is not limited to this case. For example, the subject may be a person who receives visiting nursing care.

The attribute information is any information indicating attributes of the subject. For example, the attribute information includes the subject's sex, date of birth, age, level of nursing care required, room number, and the like.

The activity function information is information pertaining to the subject's activity function. The subject's activity function includes functions related to activities of daily living (ADL) and instrumental activities of daily living (IADL). Specific examples of such functions include independence in mobility, bathing, dressing, and grooming. For example, the activity function information is expressed by a score that indicates the status of each of these functions. The score is, for example, a numerical value ranging from 0 to 55, with higher values indicating higher activity function.

The cognitive function information is information pertaining to the subject's cognitive functions. The subject's cognitive functions include communication, decision-making, comprehension, short-term memory, and long-term memory. For example, the cognitive function information is expressed by a score that indicates the status of each of these functions. The score is, for example, a numerical value ranging from 0 to 25, with higher values indicating higher cognitive function.

The nutritional status information is information pertaining to the subject's nutritional status. The subject's nutritional status includes meal content, body weight, appetite, and satisfaction with meals. For example, the nutritional status information is expressed by a score that indicates the status of each of these items. The score is, for example, a numerical value ranging from 0 to 20, with higher values indicating better nutritional status.

The health care score is a score expressing the subject's health status on a scale of 100 based on information such as the ADLs. The health care score in the present embodiment is determined based on the activity functions, cognitive functions, and nutritional status of the subject. For example, the health care score may be determined as a value that is the sum of the scores of the above-described activity functions, cognitive functions, and nutritional status. The health care score may be determined as a value that is the sum of weighted scores of the activity functions, cognitive functions, and nutritional status. The method of determining the health care score is not limited to these examples. For example, the health care score may be determined based on an index indicating the subject's independence status in addition to, or as a replacement for at least a portion of, the subject's activity functions, cognitive functions, and nutritional status. Such an index includes, for example, the level of independence in daily living.

The activity function information, cognitive status information, and nutritional status information in the subject database 220 are created or edited based on user input to the plurality of terminal apparatuses 10 or the information processing apparatus 20.

The activity functions, cognitive status, and nutritional status may be determined based on the subject's nursing care record and assessment information. Specifically, the activity functions, cognitive status, and nutritional status are determined based on 96 items extracted from the subject's nursing care record and assessment information. At least some data among this information may be automatically created or edited by a measurement apparatus such as a wearable device held by the subject. For example, at least some of the data may be determined based on medical information or information that can be acquired by various detection devices (such as information on sleep, information on physical functions obtained from video, or information on mental functions obtained from audio). The information on sleep may include respiratory rate, heart rate, sleep state, arousal, getting up, getting out of bed, and the like during sleep. The health care score in the subject database 220 is calculated based on the corresponding records (the subject's activity function information, cognitive status information, and nutritional status information).

The communication interface 23 includes at least one interface for communication with an external destination. The interface for communication may be an interface for wired communication or wireless communication. In the case of wired communication, the interface for communication may be a LAN interface or a USB, for example. In the case of wireless communication, the interface for communication may be an interface conforming to a mobile communication standard, such as LTE, 4G, or 5G, or an interface conforming to short-range wireless communication such as Bluetooth®. The communication interface 23 receives data for use in operation of the information processing apparatus 20 and transmits data resulting from operation of the information processing apparatus 20.

The functions of the information processing apparatus 20 are implemented by a processor corresponding to the controller 21 executing a program according to the present embodiment. In other words, the functions of the information processing apparatus 20 are implemented by software. The program causes a computer to function as the information processing apparatus 20 by causing the computer to execute the operations of the information processing apparatus 20. In other words, the computer functions as the information processing apparatus 20 by executing the operations of the information processing apparatus 20 in accordance with the program.

The computer in the present embodiment temporarily stores, in the main memory, the program recorded on a portable recording medium or transferred from a server, for example. The computer uses a processor to read the program stored in the main memory and executes processing with the processor in accordance with the read program. The computer may read the program directly from the portable recording medium and execute processing in accordance with the program. Each time the program is received from an external server, the computer may sequentially execute processing in accordance with the received program. Processing may be executed by an application service provider (ASP) type of service that implements functions only via execution instructions and result acquisition, without transmission of the program from an external server to the computer. Examples of the program include an equivalent to the program represented as information provided for processing by an electronic computer. For example, data that is not a direct command for a computer but that has the property of specifying processing by the computer corresponds to the “equivalent to the program”.

A portion or all of the functions of the information processing apparatus 20 may be implemented by a dedicated circuit corresponding to the controller 21. In other words, a portion or all of the functions of the information processing apparatus 20 may be implemented by hardware.

### (Operations of Information Processing Apparatus)

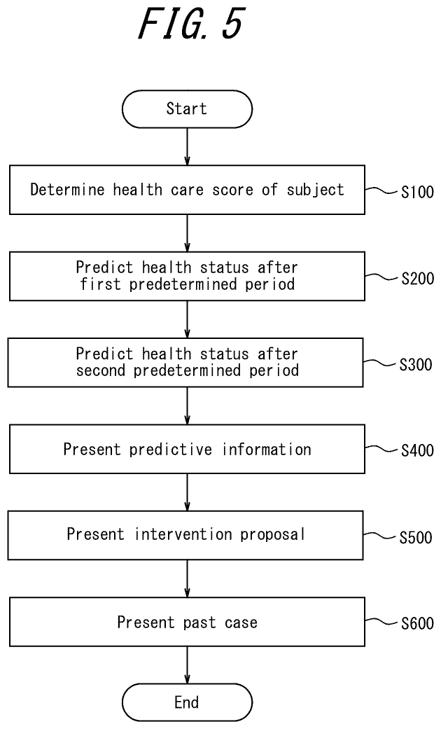

With reference to FIG. 5, operations of the information processing apparatus 20 according to the present embodiment are now described. FIG. 5 is a flowchart illustrating an example of a method executed by the information processing apparatus 20 according to the present embodiment.

Step S100: the controller 21 of the information processing apparatus 20 determines the health care score of the subject. As described above, the health care score is determined based on the activity functions, cognitive status, and nutritional status of the subject. For example, the controller 21 calculates and determines the health care score based on the activity function information, cognitive status information, and nutritional status information of the subject in the subject database 220.

Step S200: the controller 21 of the information processing apparatus 20 predicts the health status of the subject after a first predetermined period based on the health care score determined in step S100. A learning model created by machine learning using a machine learning algorithm may be used in this prediction process. The learning model may, for example, be a machine learning model constructed based on a decision tree. Examples of machine learning models constructed based on decision trees include, but are not limited to, Light GBM and XGBoost. Alternatively, the learning model may be a model generated based on a machine learning algorithm such as a Convolutional Neural Network (CNN), Recurrent Neural Network (RNN), or deep learning. Multiple regression analysis, a Kalman filter, or the like may also be used in the prediction process.

Step S300: the controller 21 predicts the health status of the subject after the second predetermined period based on the transition performance of the health status of the group to which the subject belongs, the group being determined by the health care score determined in step S100.

The group to which the subject belongs is determined based on the health care score. Specifically, the group to which the subject belongs is determined based, for example, on the score for each of the activity functions, cognitive functions, and nutritional status of the subject. In greater detail, in the present embodiment, a score for each of the activity functions, cognitive functions, and nutritional status, which constitute the subject's health care score, is evaluated on a three-tier scale of A to C, and the group to which the subject belongs is determined based on this evaluation. In other words, in the present embodiment, there are 27 groups to which subjects belong, classified from “AAA” to “CCC”. In the present embodiment, the name of each group includes these A to C evaluations. Specifically, the name of a group is, for example, represented by a character string of the three evaluations for activity function, cognitive function, and nutritional status. For example, the name of the group for which the activity function, cognitive function, and nutritional status are respectively evaluated C, A, and B is “CAB”. The evaluation method is not limited to this example, and the activity functions, cognitive functions, and nutritional status may be evaluated on a scale with two tiers, or four or more tiers. The number of group categories is not limited to 27 and may instead be 28 or more, or less than 26. The group names are not limited to the example above and may be a list of evaluation items and their respective evaluations, such as “Activity C, Cognition A, Nutrition B”.

The transition performance of the health status of a group is the past performance data for the health status of a large number of subjects. Specifically, the transition performance of the health status of a group is determined based on the group to which each subject belongs at a certain point in time (hereinafter referred to as “transition source group”) and the group to which each subject has transitioned once the second predetermined period has elapsed from a certain point in time (hereinafter referred to as “transition destination group”). The transition performance includes the probability of a group transitioning from the transition source group to the transition destination group. For example, in a case in which the transition source group of 100 subjects is “CAA”, the transition destination group of 40 subjects is “CAC”, and the transition destination group of the remaining 60 subjects is “CAA” (i.e., subjects remain in the same group), then the probability of the group transitioning from CAA to CAC is 40%, and the probability of remaining unchanged as CAA is 60%. The controller 21 predicts the health status of the subject after the second predetermined period based on this transition performance. In other words, in step S300, “predicting the health status of the subject after the second predetermined period” includes the controller 21 identifying the transition performance and transition probabilities for the group determined by the subject's health care score.

Step S400: the controller 21 presents predictive information based on the health status after the first predetermined period as predicted in step S200 and the health status after the second predetermined period as predicted in step S300.

Step S500: the controller 21 presents at least one intervention proposal that suppresses deterioration of the health status of the subject based on the transition performance of the health status of the group to which the subject belongs, the group being determined by the health care score determined in step S100. The intervention proposal includes a proposal to increase the amount of assistance and a proposal to decrease the amount of assistance. In other words, the intervention proposal includes a proposal to suppress deterioration of the subject's health status by increasing the amount of assistance and a proposal to suppress deterioration of the subject's health status by decreasing the amount of assistance.

Step S600: the controller 21 presents at least one past case of suppressed deterioration of health status of the subject based on the transition performance of the health status of the group to which the subject belongs, the group being determined by the health care score determined in step S100.

In the flowchart illustrated in FIG. 5, an example in which the information processing apparatus 20 presents predictive information, intervention proposals, and past cases is illustrated, but this example is not limiting. The information processing apparatus 20 need not present at least one of intervention proposals and past cases. In other words, the information processing apparatus 20 may skip either or both of steps S500 and S600.

Any appropriate method can be adopted here for presenting predictive information, presenting intervention proposals, and presenting past cases in step S400, step S500, and step S600. In the present embodiment, an example of presenting these pieces of information visually through a user interface is described. Such a user interface may be provided by the terminal apparatus 10 accessing the information processing apparatus 20 or a web server via browser software. Alternatively, an application according to the independence support system 1 may be installed in the terminal apparatus 10, and the user interface may be provided by the terminal apparatus 10 communicating as appropriate with the information processing apparatus 20 or an application server via the application. In this case, an authentication process or the like pertaining to the user may be performed by the terminal apparatus 10 and the information processing apparatus 20, or the terminal apparatus 10 and the web server, as appropriate.

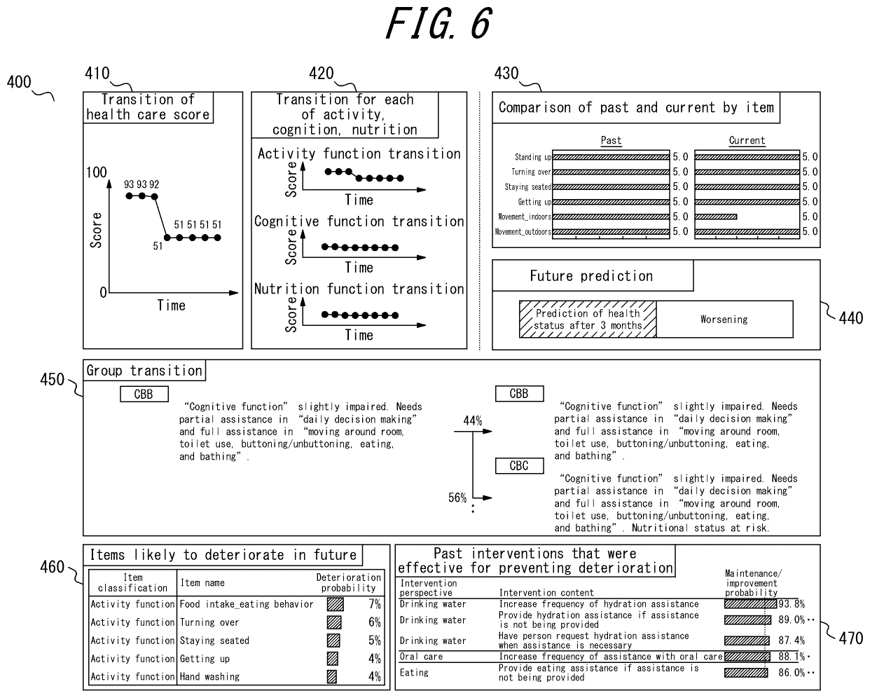

FIG. 6 illustrates an example of a user interface displayed by the output interface 15 of the terminal apparatus 10. FIG. 6 illustrates a user interface 400, for a certain subject, provided to a user of a residential facility as an example. As illustrated in FIG. 6, the user interface 400 includes objects 410 to 470. The user interface 400 includes information pertaining to the above-described predictive information, intervention proposals, and past cases for each subject.

The object 410 is an object that contains information pertaining to the subject's health care score. Such an object may include a transition graph or the like of the subject's past and current health care scores. In the example illustrated in FIG. 6, the object 410 includes a transition graph in which the horizontal axis represents time and the vertical axis represents the value of the health care score. The object 410 enables the user easily to grasp the current and past values of the subject's health care score and their transition.

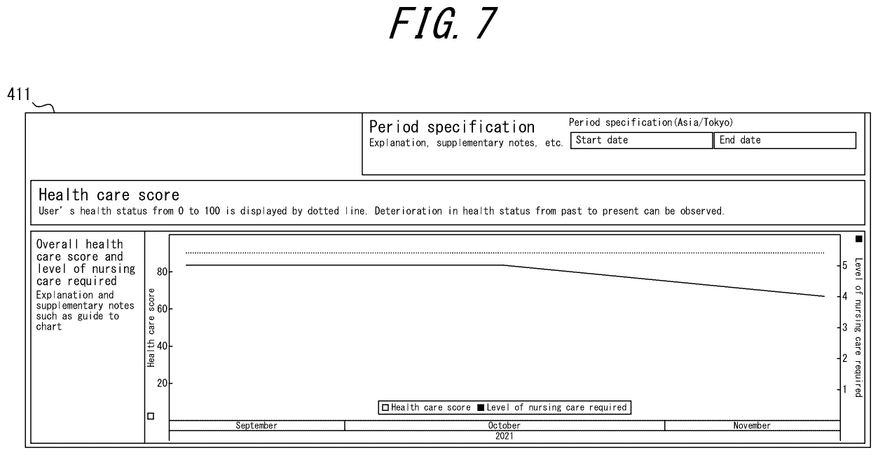

FIG. 7 illustrates a variation of the object 410. The object 411 illustrated in FIG. 7 is an object that includes information pertaining to the subject's health care score. The object 411 includes a transition graph in which the horizontal axis represents time and the vertical axis represents the value of the health care score. The object 411 also includes a transition graph of the subject's level of nursing care required, in which the horizontal axis represents time and the vertical axis represents the level of nursing care required. The object 410 enables the user easily to grasp the current and past values of the subject's level of nursing care required and their transition, in addition to the current and past values of the subject's health care score and their transition.

The object 420 is an object that contains information pertaining to the respective scores for activity functions, cognitive functions, and nutritional status of the subject. Such an object may include a transition graph or the like of each score for the past and present activity functions, cognitive functions, and nutritional status of the subject. In the example illustrated in FIG. 6, the object 420 includes transition graphs in which the horizontal axis represents time and the vertical axis represents the value of the scores for activity functions, cognitive functions, and nutritional status. The object 420 enables the user easily to grasp the current and past values of the scores, and their transition, for the activity functions, cognitive functions, and nutritional status of the subject.

The object 430 is an object that includes information pertaining to each item of the subject's activity functions. Such an object may include a bar graph or the like pertaining to each item of the subject's past and present activity functions. In the example illustrated in FIG. 6, the object 430 includes a bar graph in which past and present evaluation values are arranged side by side for each item of the activity functions. The object 430 enables the user easily to compare the current and past evaluation values of each item of the subject's activity functions.

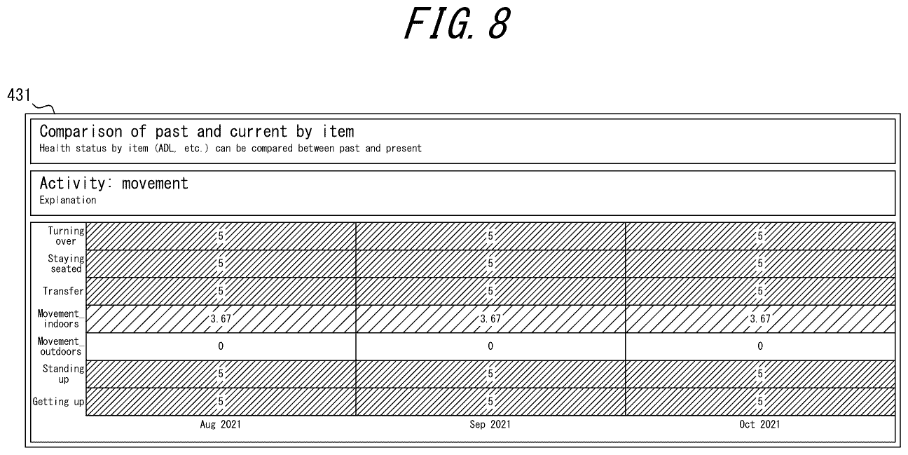

FIG. 8 illustrates a variation of the object 430. The object 431 illustrated in FIG. 8 is an object that includes information pertaining to each item of the subject's activity functions. The object 431 includes a heat map in which the horizontal axis represents time and the vertical axis represents each item of the activity functions. The object 431 enables the user easily to compare the change in evaluation values from the past to the present for each item of the subject's activity functions.

The object 440 is an object that includes predictive information based on the subject's health status after the first predetermined period. Such an object may include predictive information such as “maintaining” or “worsening”. In the example illustrated in FIG. 6, the object 440 presents “worsening” as the predictive information for the subject after the first predetermined period (for example, after 3 months). The predictive information indicated by the object 440 is not limited to this example and may, for example, be predictive information indicated in three tiers (such as “improving”, “maintaining”, and “worsening”), four tiers (such as “improving”, “maintaining”, “possibly worsening”, and “worsening”), or more than four tiers. The predictive information may be numerical information, such as the predicted value of the health care score after the first predetermined period. Alternatively, the predictive information may be a combination of text information such as “maintaining” or “worsening” and numerical information such as the predicted value of the health care score. The object 440 enables the user easily to grasp a prediction of the subject's health status after the first predetermined period.

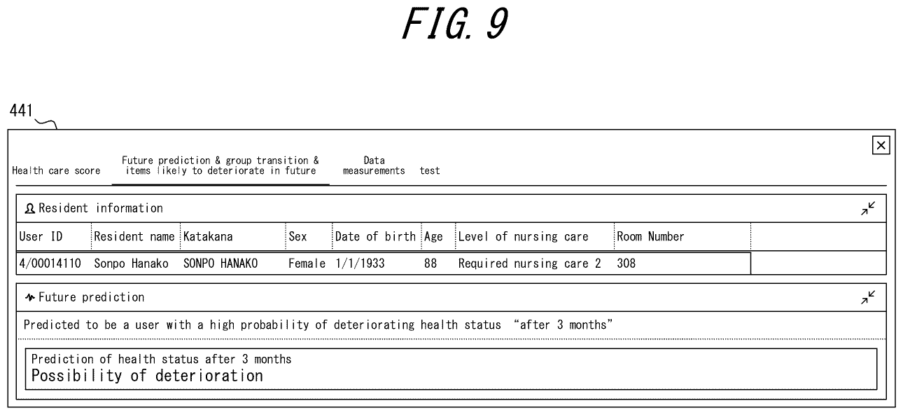

FIG. 9 illustrates a variation of the object 440. The object 441 illustrated in FIG. 9 is an object that includes predictive information based on the subject's health status after the first predetermined period. The object 441 presents information about the subject and presents “possibly worsening” as the predictive information for the subject after the first predetermined period (for example, after 3 months). The object 411 also enables the user easily to grasp a prediction of the subject's health status after the first predetermined period.

The object 450 is an object that includes predictive information based on the subject's health status after the second predetermined period. The health status of the subject after the second predetermined period is predicted based on the transition performance of the health status of the group to which the subject belongs, the group being determined by the health care score. Therefore, in the present embodiment, the information included in the object 450 is also referred to as the “group transition”. The object 450 illustrated in FIG. 6 includes information on the transition source group, transition destination groups, and transition probability as the group transition. Specifically, in the example illustrated in FIG. 6, the transition source group is “CBB”. The object 450 may include a description of the transition source group. For example, the description of “CBB” is “‘Cognitive function’ slightly impaired. Needs partial assistance in ‘daily decision making’ and full assistance in ‘moving around room, toilet use, buttoning/unbuttoning, eating, and bathing’”. The transition destination groups included in the object 450 are “CBB” and “CBC”. The object 450 may include a description of the transition destination groups. The description of “CBB” is the same as above. The description of “CBC” is “‘Cognitive function’ slightly impaired. Needs partial assistance in ‘daily decision making’ and full assistance in ‘moving around room, toilet use, buttoning/unbuttoning, eating, and bathing’. Nutritional status at risk”. The probability of transitioning to the first candidate transition destination group (CBB) is 44%. On the other hand, the probability of transitioning to the second candidate transition destination group (CBC) is 56%. These transition probabilities are statistical information determined based on the past transition performance of the health status of the subjects belonging to each group. In other words, the transition probability is a performance value determined based on past performance. That is, the object 450 enables the user easily to grasp statistical information on the future health status of the subject.

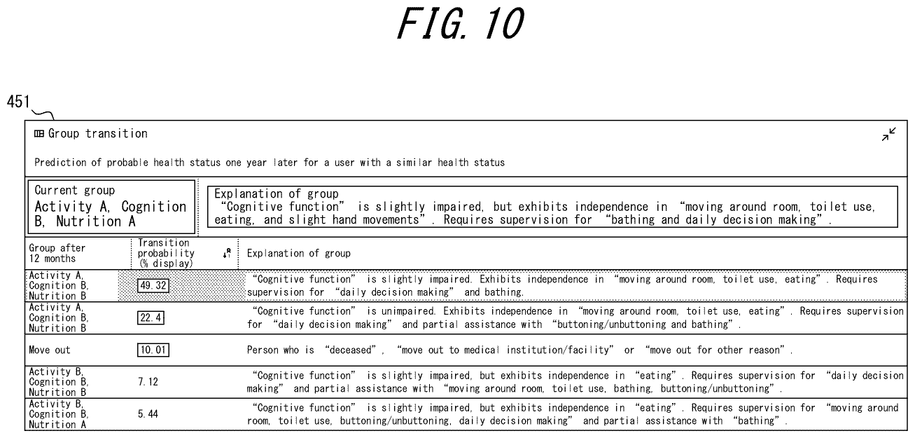

FIG. 10 illustrates a variation of the object 450. The object 451 illustrated in FIG. 10 is an object that includes predictive information based on the subject's health status after the second predetermined period. The health status of the subject after the second predetermined period is predicted based on the transition performance of the health status of the group to which the subject belongs, the group being determined by the health care score. The object 451 includes information on the transition source group, transition destination groups, and transition probability as the group transition. The information on the transition source group included in the object 451 is “Activity A, Cognition B, Nutrition A”. The object 451 may include a description of the transition source group. For example, the description of “Activity A, Cognition B, Nutrition A” is “‘Cognitive function’ is slightly impaired, but exhibits independence in ‘moving around room, toilet use, eating, and slight hand movements’. Requires supervision for ‘bathing and daily decision making’”. The transition destination groups included in the object 451 are “Activity A, Cognition B, Nutrition A”, “Activity A, Cognition B, Nutrition B”, “Move out”, and the like. The object 451 may include a description of these groups. The transition probabilities for the group transitions are 49.32%, 22.4%, and 10.01% respectively for “Activity A, Cognition B, Nutrition A”, “Activity A, Cognition B, Nutrition B”, and “Move out”, as illustrated in FIG. 10. This statistical information enables the user easily to grasp the transition of the subject's health status.

The object 460 is an object that includes predictive information based on the subject's health status after the first predetermined period. As such predictive information, the object 460 includes information on functional items that might deteriorate for the subject after the first predetermined period. In other words, the predictive information includes a functional item that might deteriorate after the first predetermined period. The functional items that might deteriorate after the first predetermined period may be determined based on the performance of functional items that deteriorated after the first predetermined period for persons belonging to the same group as the subject. That is, the functional items that might deteriorate after the first predetermined period may be determined based on the transition performance of the health status of the group to which the subject belongs, the group being determined by the health care score. The functional items that might deteriorate after the first predetermined period may be determined based on the health status of the subject after the first predetermined period, as predicted based on the health care score determined in step S100.

The aforementioned information on the functional items included in the object 460 includes the classification of the corresponding functional item (hereinafter referred to as “item classification”), the item name, and the deterioration probability. The item classification, item name, and deterioration probability are, for example, “activity function”, “food intake_eating behavior”, and “7%”. As illustrated in FIG. 6, the object 460 may include a bar graph of the deterioration probability next to the numerical value of the deterioration probability. As also illustrated in FIG. 6, such functional items may be arranged in order starting with the highest deterioration probability. The object 460 enables the user easily to grasp items that are particularly likely to deteriorate in the future within the subject's future health status.

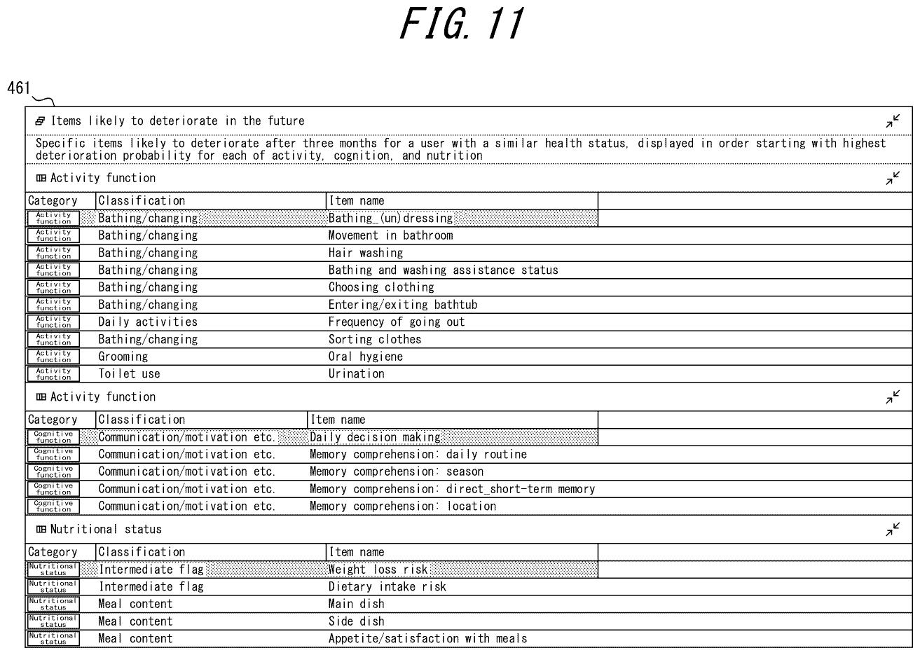

FIG. 11 illustrates a variation of the object 460. As predictive information, the object 461 includes information on functional items that might deteriorate for the subject after the first predetermined period. The aforementioned information on the functional items included in the object 461 is classified by activity function, cognitive function, and nutritional status. The aforementioned information on the functional items included in the object 461 includes the category of the functional item, the item classification, and the item name. As also illustrated in FIG. 11, such functional items may be arranged in order starting with the highest deterioration probability for each of activity function, cognitive function, and nutritional status. The object 461 enables the user easily to grasp items that are particularly likely to deteriorate in the future within the subject's future health status.

The object 470 is an object that includes information on at least one intervention proposal that suppresses deterioration of the health status of the subject. The intervention proposal is determined based on the transition performance of the health status of the group to which the subject belongs, the group being determined by the health care score of the subject. In the example illustrated in FIG. 6, the information on the intervention proposal included in the object 470 includes an intervention perspective, intervention content, and a maintenance/improvement probability. The intervention perspective, intervention content, and maintenance/improvement probability are, for example, “drinking water”, “increase frequency of hydration assistance”, and “93.8%”. As illustrated in FIG. 6, the object 470 may include a bar graph corresponding to the numerical value of the maintenance/improvement probability. The object 470 enables the user easily to grasp intervention proposals that suppress the deterioration of the subject's health status.

As described above, according to the present embodiment, the information processing apparatus 20 determines the health care score of the subject, predicts the health status after the first predetermined period based on the health care score, predicts the health status of the subject after the second predetermined period, and presents predictive information. In other words, according to the present embodiment, technology related to independence support is improved in that the subject's health care score can be used to present predictive information from multiple different perspectives. Furthermore, since the health status after the second predetermined period is predicted based on the transition performance of the health status for the group, the user can easily grasp the past performance in similar situations.

According to the present embodiment, the predictive information presented by the information processing apparatus 20 also includes functional items that might deteriorate after the first predetermined period. Accordingly, the user can easily grasp items that might deteriorate in the future within the subject's future health status. Furthermore, the predictive information presented by the information processing apparatus 20 may include the deterioration probability of the functional items, in which case the user can quantitatively grasp the degree to which each functional item may deteriorate.

In the present embodiment, the first predetermined period is a shorter period than the second predetermined period. The first predetermined period and the second predetermined period may be 3 months and 1 year, respectively, but this example is not limiting. By the first predetermined period being shorter than the second predetermined period, the user can grasp the subject's future health status from both a relatively short-term perspective and a relatively long-term perspective.

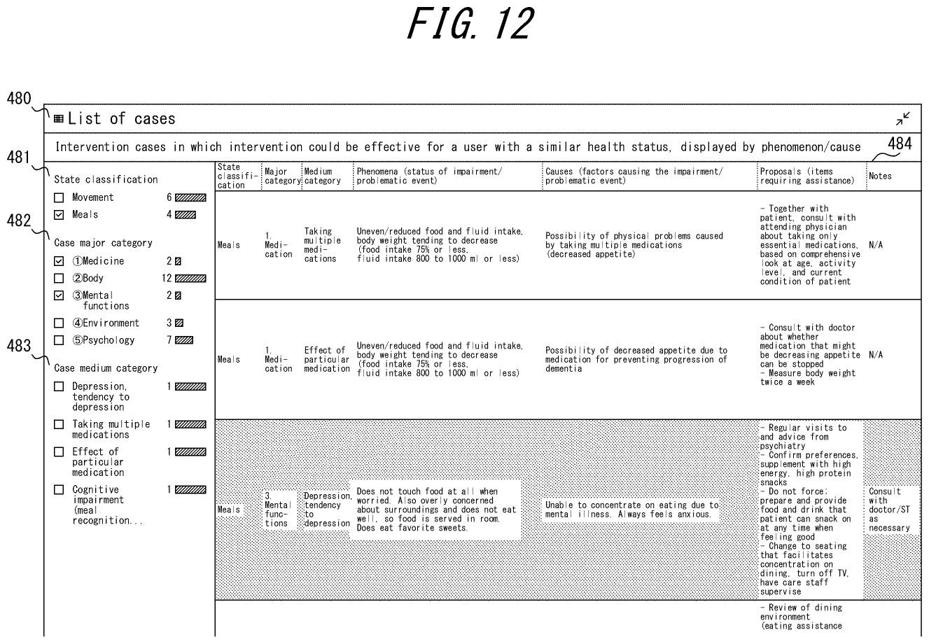

An example in which the user interface 400 includes the objects 410 to 470 has been illustrated in the present embodiment, but this configuration is not limiting. For example, the user interface 400 may include an object that includes at least one past case of suppressed deterioration of health status instead of or in addition to the object 470. The past case is determined based on the transition performance of the health status of the group to which the subject belongs, the group being determined by the health care score of the subject. FIG. 12 illustrates an example of the object 480. The object 480 includes filtering objects 481 to 483 by state classification, case major category, and case medium category. The filtering objects 481 to 483 can be used for appropriate filtering of past intervention cases. The object 480 includes a table 484 of past intervention cases. The table 484 includes the state classification, major category, medium category, phenomena (status of the impairment/problematic event), causes (factors causing the impairment/problematic event), proposals (items requiring assistance), and notes. In other words, the table 484 lists various information about past intervention cases. In a case in which this object 480 is included in the user interface 400, the user can easily grasp past cases in which deterioration of the subject's health status was suppressed.

The intervention proposals and past cases may include uninsured services. Examples of uninsured services include voluntary guidance or training (diet instruction, exercise instruction, cognitive function training, or the like), and voluntary programs to prevent a decline in cognitive function.

In the example illustrated in the present embodiment, the group to which the subject belongs is determined based on a score for each of the activity function, cognitive function, and nutritional status of the subject (i.e., the individual scores that constitute the health care score), but this configuration is not limiting. The group to which the subject belongs may be determined directly from the health care score. In this case, the group to which the subject belongs may be classified into any tier based on the numerical value of the health care score, for example.

Although the present disclosure is based on embodiments and drawings, it is to be noted that various changes and modifications will be apparent to those skilled in the art based on the present disclosure. Therefore, such changes and modifications are to be understood as included within the scope of the present disclosure. For example, the functions and the like included in the various units and steps may be reordered in any logically consistent way. Furthermore, a plurality of units and steps may be combined into one, or a single unit or step may be divided.

### REFERENCE SIGNS LIST

1 Independence support system
10 Terminal apparatus
11 Controller
12 Memory
13 Communication interface
14 Input interface
15 Output interface
20 Information processing apparatus
21 Controller
22 Memory
23 Communication interface
30 Network
220 Subject database
400 Interface
410, 420, 430, 440, 450, 460, 470, 480 Object
411, 431, 441, 451, 461 Object
481 to 483 Filtering object
484 Table

1. An independence support method to be executed by an information processing apparatus, the independence support method comprising:
determining a health care score of a subject;
predicting a health status of the subject after a first predetermined period based on the health care score;
predicting a health status of the subject after a second predetermined period based on a transition performance of a health status of a group to which the subject belongs, the group being determined by the health care score; and
presenting predictive information based on the health status after the first predetermined period and the health status after the second predetermined period.

2. The independence support method according to claim 1, wherein the group is determined by a score for each of an activity function, a cognitive function, and a nutritional status of the subject.

3. The independence support method according to claim 1, wherein the predictive information includes a functional item that might deteriorate after the first predetermined period.

4. The independence support method according to claim 3, wherein the predictive information includes a probability of deterioration of the functional item.

5. The independence support method according to claim 1, wherein the first predetermined period is a shorter period than the second predetermined period.

6. The independence support method according to claim 1, wherein the health care score is determined based on an activity function, a cognitive function, and a nutritional status of the subject.

7. The independence support method according to claim 1, further comprising presenting at least one intervention proposal that suppresses deterioration of a health status of the subject, the intervention proposal being determined based on the transition performance of the health status of the group.

8. The independence support method according to claim 1, further comprising presenting at least one past case of suppressed deterioration of a health status, the past case being determined based on the transition performance of the health status of the group.

9. An information processing apparatus comprising a controller, wherein
the controller is configured to
determine a health care score of a subject,
predict a health status of the subject after a first predetermined period based on the health care score,
predict a health status of the subject after a second predetermined period based on a transition performance of a health status of a group to which the subject belongs, the group being determined by the health care score, and
present predictive information based on the health status after the first predetermined period and the health status after the second predetermined period.

10. A system comprising the information processing apparatus according to claim 9 and a terminal apparatus, wherein
the information processing apparatus is configured to transmit the predictive information to the terminal apparatus, and
the terminal apparatus is configured to output the received predictive information.

11. A non-transitory computer readable medium storing a program configured to cause a computer to execute operations comprising:
determining a health care score of a subject;
predicting a health status of the subject after a first predetermined period based on the health care score;
predicting a health status of the subject after a second predetermined period based on a transition performance of a health status of a group to which the subject belongs, the group being determined by the health care score; and
presenting predictive information based on the health status after the first predetermined period and the health status after the second predetermined period.
